(19) United States
(12) Patent Application Publication
(10) Pub. No.: US 2019/0195820 A1
(43) Pub. Date: Jun. 27, 2019

(54) RELATIVE HUMIDITY SENSOR CALIBRATION

(71) Applicant: TEXAS INSTRUMENTS INCORPORATED, Dallas, TX (US)

(72) Inventors: Andrea FORNASARI, Pavia (IT); Raffaele BOI, Pavia (IT); Alberto Luca DEMARZIANI, Vigevano (IT); Massimo MASCOTTO, Milan (IT); Emanuele QUARTIERI, Massalengo (IT); Piero MALCOVATI, Pavia (IT); Stefano MANGIAROTTI, San Martino Siccomario (IT)

(21) Appl. No.: 15/852,335

(22) Filed: Dec. 22, 2017

(51) Int. Cl. G01N 27/22; G01N 33/18; G05D 22/02

(52) U.S. Cl. CPC G01N 27/223; G05D 22/02; G01N 27/228; G01N 33/18

(57) ABSTRACT

An integrated humidity and temperature sensor includes a humidity sensor configured to output a signal corresponding to a first humidity value at a first time and a second humidity value at a second time, a temperature sensor configured to output a signal corresponding to a first temperature at the first time and a second temperature at the second time, a heating element configured to raise a temperature of the humidity sensor and the temperature sensor between the first time and the second time, and a processor device configured to determine a drift value of the humidity sensor according to the first and the second temperatures and the first and the second humidity values. The processor device is also configured to adjust the humidity sensor by the drift value. Adjustment of the humidity sensor can be a shift of an output of the humidity sensor by the drift value.

### BACKGROUND

Relative humidity (RH) sensors, similar to other sensors, even if ideally calibrated in production, can deviate from ideal behavior over time and/or if exposed to conditions outside a normal operative range (e.g., high temperature, high humidity, or contaminant gases). Sensor response is a function of target input, but is also effected by cross-sensitivities, such as environmental conditions (typically temperature, but, depending on the sensor, other parameters such as pressure or gas concentration). In addition to typical non-ideal conditions, some sensors suffer from aging (i.e., a drift or offset in accuracy due to time in use and/or to exposure to contaminants). In short, sensors accumulate offset with aging and this offset corrupts measurement.

### SUMMARY

In one aspect, a method detects whether sensor aging occurs (degradation of sensor accuracy due to exposition to time and/or extreme environmental conditions) and if so, compensates for the aging (recalibrating the sensor). In one example, a method includes checking a consistency of sensor behavior as a function of a secondary variable (for example, temperature, pressure, gas concentration). Example methods apply to many classes of sensors (e.g., relative humidity sensors).

In accordance with one example, a method of calibrating a humidity sensor includes measuring a first humidity value using a humidity sensor at a first time; measuring a first value of a secondary parameter, indicative of a value of the secondary parameter at the humidity sensor, at the first time; altering the secondary parameter; measuring a second humidity value using the humidity sensor at a second time; measuring a second value of the secondary parameter at the second time; and determining a drift value of the humidity sensor according to the first and the second values of the secondary parameter and the first and the second humidity values. The humidity sensor can then be adjusted by the drift value.

In other examples, the secondary parameter is temperature, and the altering of the secondary parameter is a raising of the temperature. In another example, adjusting the humidity sensor is a shifting of an output of the humidity sensor by the drift value. In still another example, a certain drift relationship compares the actual dependency of the sensor by temperature with a theoretical behavior and automatically adjusts the reading offset of the sensor to better match an ideal output. In further examples, a method of calibrating a humidity sensor uses the humidity sensor, which is a digital humidity and temperature sensor, including a humidity sensor, a temperature sensor and a heating element.

In accordance with another example, a method of calibrating a humidity sensor includes measuring a first humidity value using a humidity sensor at a first time; measuring a first temperature indicative of a temperature of the humidity sensor at the first time; raising a temperature of the humidity sensor; measuring a second humidity value using the humidity sensor at a second time; measuring a second temperature indicative of a temperature of the humidity sensor at the second time; determining a drift value of the humidity sensor according to the first and the second temperatures and the first and the second humidity values; and adjusting the humidity sensor by the drift value.

In accordance with a further example, an integrated humidity and temperature sensor includes a humidity sensor configured to output a signal corresponding to a first humidity value at a first time and a second humidity value at a second time; a temperature sensor configured to output a signal corresponding to a first temperature at the first time and a second temperature at the second time; a heating element configured to raise a temperature of the humidity sensor and the temperature sensor between the first time and the second time; and a processor device configured to determine a drift value of the humidity sensor according to the first and the second temperatures and the first and the second humidity values.

In accordance with a still further example, an apparatus includes a humidity sensor configured to output a signal corresponding to a humidity value; a temperature sensor configured to output a signal corresponding to a temperature; a heating element configured to raise a temperature of the humidity sensor and the temperature sensor; and a processor device in communication with the humidity sensor, the temperature sensor, and the heating element. The processor device is configured to measure a first humidity value at a first time; measure a first temperature at the first time; raise a temperature within the apparatus; measure a second humidity value at a second time; measure a second temperature at the second time; and determine a drift value of the humidity sensor according to the first and the second temperatures and the first and the second humidity values. The processor device can also be configured to then adjust the humidity sensor by the drift value.

### BRIEF DESCRIPTION OF THE DRAWINGS

For a detailed description of various examples, reference will now be made to the accompanying drawings in which:

FIG. 1 illustrates a cross-section of one aspect of an integrated humidity and temperature sensor, in accordance with various examples;

FIG. 2 illustrates a diagrammatic view of an integrated humidity and temperature sensor, in accordance with various examples;

FIG. 3 illustrates a flow chart of a method of sensor calibration, in accordance with various examples; and

FIGS. 4A-4C illustrate before and after results of an example implementation of a method of calibrating a relative humidity sensor using an integrated humidity and temperature sensor; where FIG. 4A illustrates a drift (Error RH[%]) of 32 fresh sensor units; FIG. 4B illustrates the drift due to simulated aging of the 32 sensor units; and FIG. 4C illustrates a drift of the 32 sensor units after recalibration.

### DETAILED DESCRIPTION

Specific aspects and examples will now be described in detail with reference to the accompanying figures. Like elements in the various figures are denoted by like reference numerals for consistency. In the following detailed description, numerous specific details are set forth in order to provide a more thorough understanding. However, it will be apparent to one of ordinary skill in the art that the certain described aspects may be practiced without these specific details. In other instances, well-known features have not been described in detail to avoid unnecessarily complicating the description.

Certain terms are used throughout the following description and claims to refer to particular system components. As one skilled in the art will appreciate, different parties may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct wired or wireless connection. Thus, if a first device couples to a second device, that connection may be through a direct connection or through an indirect connection via other devices and connections. The recitation “based on” is intended to mean “based at least in part on.” Therefore, if X is based on Y, X may be a function of Y and any number of other factors.

A typical sensor, for example, a relative humidity (RH) sensor, operates stably within a recommended normal range (for a RH sensor, as a function of relative humidity and temperature). Long term exposure to conditions outside a normal range (for example, 85%RH/85degC) may temporarily offset the relative humidity signal. When exposure outside a normal range is limited in time, the sensor will slowly return to factory calibration. But, prolonged exposure to extreme conditions may accelerate aging, and increase long term drift or offset (expressed in %RH/yr).

Generally, sensor aging is caused by issues related to a chemistry of the sensor. In a relative humidity sensor, the sensing element is a polymer (a polyimide—PI—or one of its derivatives), able to absorb moisture as a function of the relative humidity in air. A resulting change in permittivity (εₚ) can be easily detected by a capacitance to a digital converter. Prolonged exposure to extreme conditions can alter the sensor chemistry, causing a physiochemical change leading to drift in sensor performance.

Relative humidity (RH) is defined as the ratio of the partial pressure of water vapor (e_{w}) to the saturated vapor pressure of water at a given temperature (e_{w}^{h}). While the partial pressure of water vapor can be considered as a function of the moisture concentration, the saturated vapor pressure is function of temperature only.

Accordingly, the following relationship can be shown:

$$e {*}_{W} = a \cdot e ( \frac{bT}{c + T} ) \qquad ( 1 )$$

where an accuracy of the formula (1) with a=6.1121, b=17.123, c=234.95, is better than 0.15% in the [0,100] degC range. Constants a, b and c, above, would vary depending on pressure, and the above values consider a standard, sea-level pressure of one standard atmosphere.

A strong dependency on temperature exists. For example, it requires less water vapor to attain high relative humidity at low temperatures; while requiring more water vapor to attain high relative humidity in warm or hot air. As a general rule, for 1degC variation of temperature, the relative humidity changes by 5% of initial value (so, a 40% relative humidity at 30degC becomes about 38% relative humidity at 31degC, or 42% relative humidity at 29degC).

In one aspect, two consecutive humidity and temperature measurements (at different temperatures) are taken, and a drift value of the humidity sensor is determined according to the two temperatures and the two humidity values. From the determination of the drift, a user can understand if the sensor is aged (diagnostic) and can then compensate (recalibrate) the sensor based upon the drift value. Accordingly, a user can verify, in the field or in the factory, if a sensor follows the above relationship, by forcing and carefully measuring a temperature parameter associated with relative humidity. In one example, a user would change a temperature of the sensor fast enough to assume a constant moisture concentration in the air before and after the temperature rise.

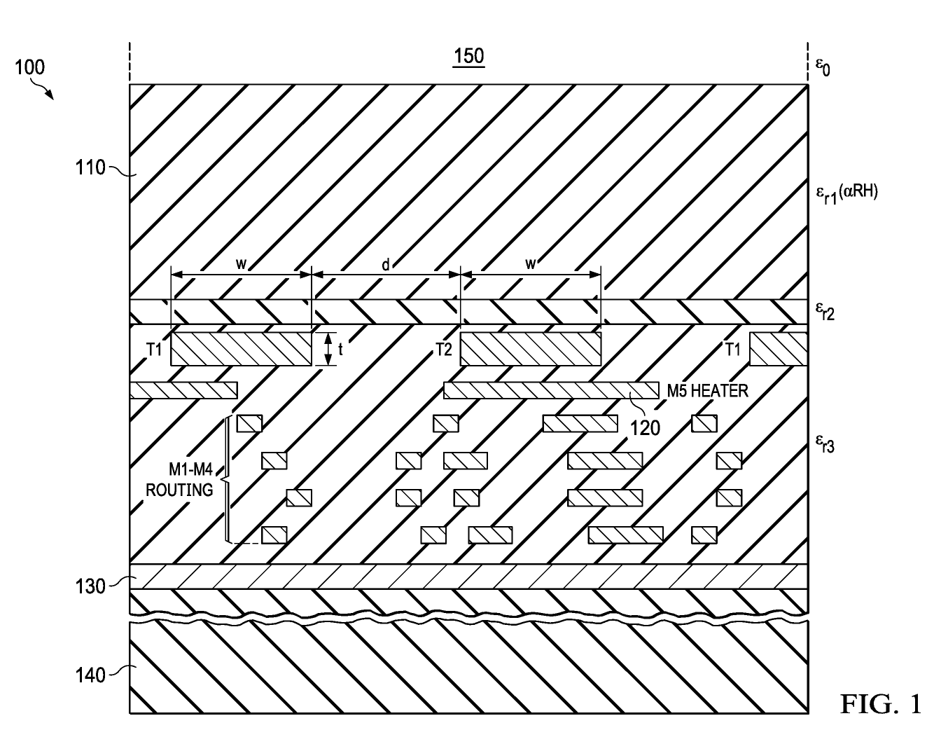

In one example, FIG. 1 illustrates a cross-section of an integrated humidity and temperature sensor 100 including a relative humidity sensing element 110, a heating element 120, a temperature sensing element 130 and bulk silicon circuit 140. The relative humidity sensing element 110 is a polyimide, and is located outside of the silicon circuit 140. The humidity sensing element 110 can absorb moisture as a function of relative humidity in air 150. The heating element 120 is in a form of a resister used to raise a temperature between the two consecutive humidity and temperature measurements. In other examples, a resistor could be placed as close as possible to the relative humidity sensing element 110.

In the example integrated humidity sensor 100, capacitance change is detected by a two terminal (T1 and T2) fringe capacitor. Width (w) and distance (d) of fingers have been optimized to maximize the sensitivity to εᵣ₁. The heating element 120 is a serpentine resistor (to grant heating uniformity) designed directly below the relative humidity sensing element 110. The temperature sensing element 130 is based on bipolar technology and is located in an active region.

Integrated humidity sensors are generally known to include heating elements. However, a purpose of the heating element is to assist sensor recovery from condensation, or to reset the sensor when exposed to a volatile organic compound (VOC) (e.g., gas trapped in the sensor modifies sensor behavior, where exposing the sensor to high temperature degases the VOC). In one example, an existing heating element of an integrated humidity sensor can also be used to raise sensor temperature between the two consecutive humidity and temperature measurements.

In one aspect, drift or offset is determined by calculating a numerator over a denominator. In this aspect, the numerator is a first value minus the second humidity value. The first value is the first humidity value multiplied by a second value, where the second value includes a difference between the first temperature and the second temperature. In this aspect, the denominator includes the second value.

In another aspect, drift or offset is presented by the following relationship:

$$drift = \frac{{RHoM}^{eb} ( \frac{{T}_{0}}{c + {T}_{0}} - \frac{{T}_{1}}{c + {T}_{1}} ) - {RH}_{1 M}}{{e}^{b} ( \frac{{T}_{0}}{c + {T}_{ 0}} - \frac{{T}_{1}}{c + {T}_{1}} ) - 1} \qquad ( 2 )$$

where RH_{oM} and RH_{1M} are measured relative humidity, respectfully, at the T₃ and T₁ temperatures (expressed in degC). Again, b=17.123 and c=234.95.

The above relationship for drift does not assume that the same amount of water (expressed in grams per cubic meter) is in the air before and after the heating phase (i.e., at a time of, or before and after the time of, two consecutive humidity and temperature measurements, where temperature is raised between the two measurements). One thing considered constant before and after the heating phase is pressure, which is considered in the relationship for drift based upon a dew point temperature constant (where dew point is defined as the temperature to which air must be cooled to become saturated with water vapor (i.e., when moisture starts to condense)).

The above relationship for drift can be loaded into a microcontroller, saved in memory and implemented by processor. Alternatively, the drift relationship can be hardwire implemented in a logic state machine of an integrated circuit.

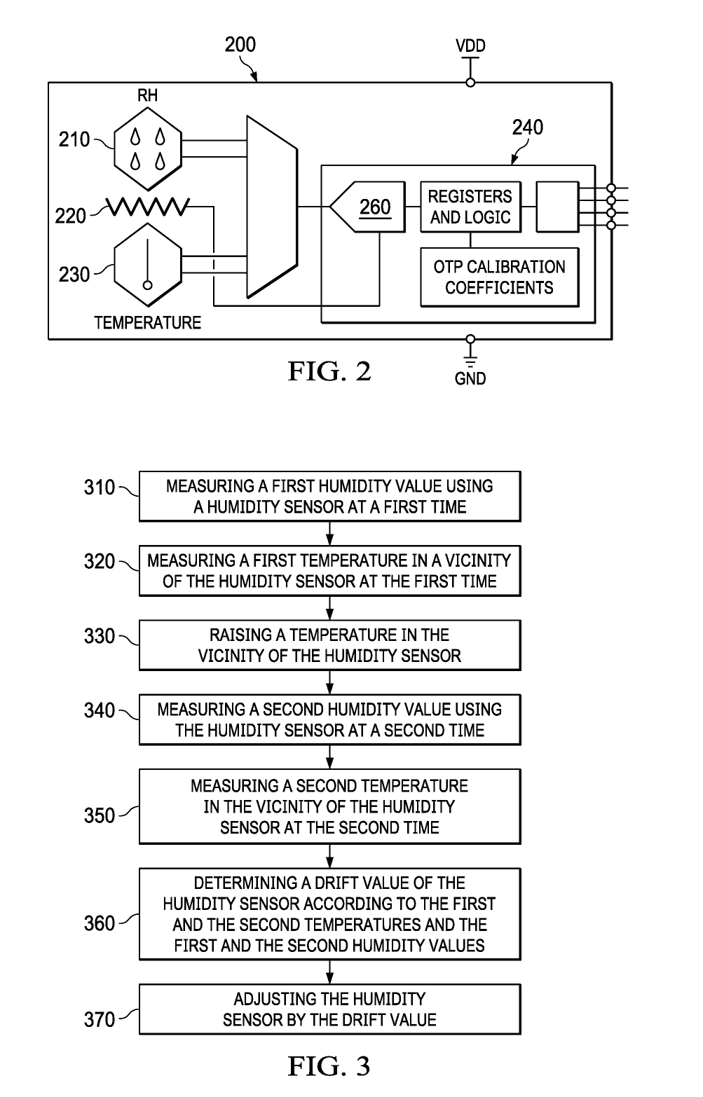

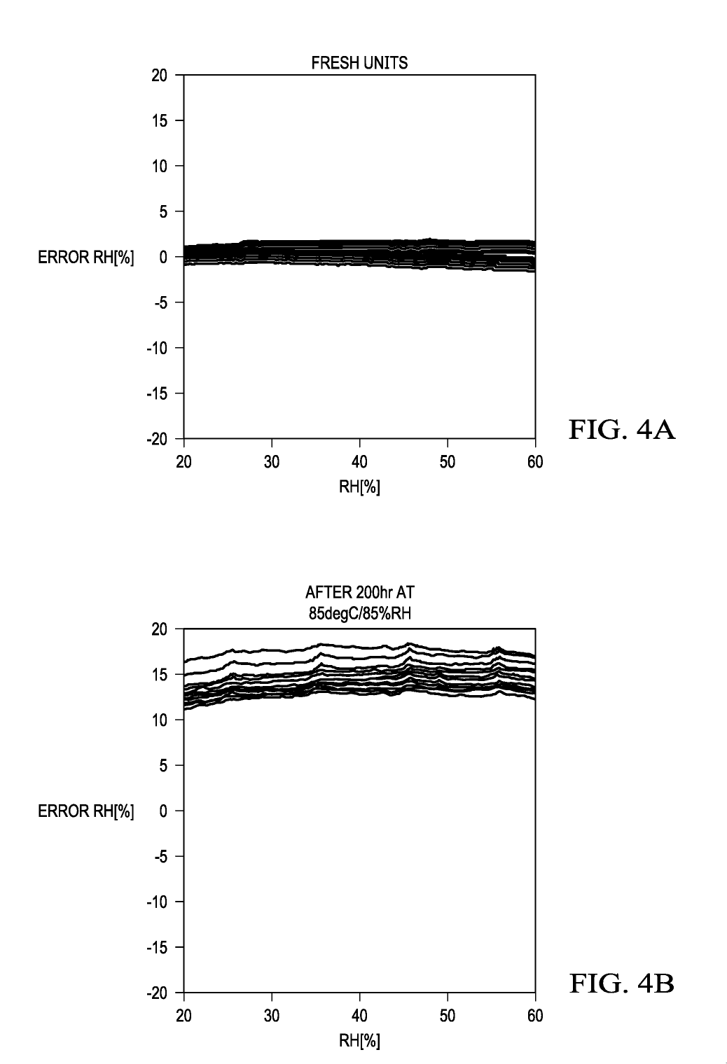

FIG. 2 illustrates an example integrated humidity and temperature sensor 200, including a relative humidity sensing element 210, a heating element 220, a temperature sensing element 230 and a processor device 240. The processor device 240 is in electrical communication with the relative humidity sensing element 210, the heating element 220, and the temperature sensing element 230, which includes electrical communication of these components to a processing element or circuit 260. The processor device 240 is configured to implement the above-identified, formula (2), drift relationship, and to execute the methods of sensor calibration described below with respect to FIG. 3. Those skilled in the art will recognize that other configurations of the processor device 230 are possible, and that such a processor can comprise a fixed purpose hard wired platform or can comprise a partially or wholly programmable platform. These architectural options are known and understood in the art and require no further description here.

In another aspect, a method of calibrating a sensor is provided. In one example, the sensor is a relative humidity sensor. The relative humidity sensor is monitored as a function of a secondary variable. In one example, the secondary variable is temperature. In this aspect, the method determines any deviation from a proper, or ideal, result, and then automatically adjusts the sensor offset to better match the ideal result.

FIG. 3 illustrates an example method of calibrating a relative humidity sensor. In step 310, a first humidity value, RH_{oM}, is measured using a humidity sensor at a first time, to. At step 320, a first temperature, Tₒ, is measured in a vicinity of the humidity sensor at the first time, t₀. In one aspect of the disclosure, the temperature step is approximately 60degC and time approximately 20 seconds.

At step 330, a temperature is raised in the vicinity of the humidity sensor. In one aspect, only a few seconds of heating is necessary. In this heating step, a particular temperature step profile is not required. The temperature step profile can be fixed time with variable temperature step, or fixed temperature step with variable time.

For example, to increase the humidity sensor temperature by fixed time with variable temperature step, the heating element is switched on for a certain (fixed) period of time, and a final temperature is measured at the end of the fixed period of time, regardless of the amount of temperature rise (e.g., the temperature rise, step, or delta, is variable). In this aspect, the particular temperature rise is related to ambient temperature, the printed circuit board (PCB) design, the application case material/shape, ventilation, etc. To increase the humidity sensor temperature by fixed temperature step with variable time, the heating element is switched on and the temperature is monitored until a certain temperature rise, or delta, is reached. In this aspect, the duration of time required to attain the fixed temperature delta is related to the ambient temperature, the PCB design, the application case material/shape, ventilation, etc. In certain examples of fixed temperature step with variable time, it may not be possible to precisely know, in advance, the amount of heating time necessary to attain the certain temperature step, or rise. Each approach works well, and selection of which approach can be based upon application convenience. For examples, for a small PCB, the fixed time with variable temperature step approach may be convenient, as power consumption is better controlled and a sufficient temperature delta is easily achieved

At step 340, a second humidity value, RH_{1M}, is measured using the humidity sensor at a second time, t₁, after temperature is raised. In one example, the second humidity value is measured immediately upon temperature rise, and as quickly as possible relative to the first time, t₀, to facilitate constant pressure between the first time, t₀, and the second time, t₁. At step 350, a second temperature, T₁, is measured in the vicinity of the humidity sensor at the second time, t_{16l .}

At step 360, a drift or offset value of the humidity sensor is determined according to the first and the second temperatures and the first and the second humidity values. In one example, the drift or offset value of the humidity sensor is determined according to the above-identified, formula (2), drift relationship. In one aspect of this drift relationship, constant b=17.123 and c=234.95. In another aspect, recalibration is recommended when a respective sensor has a determined drift of greater than +/−3% Error RH.

At step 370, the humidity sensor is recalibrated by adjusting the humidity sensor. In one example, the humidity sensor is adjusted by the drift value. In another example, adjusting the humidity sensor is a shifting of an output of the humidity sensor by the drift value.

In another aspect, the method of calibrating a relative humidity sensor uses an integrated humidity and temperature sensor. In a further aspect, raising the temperature in the vicinity of the humidity sensor is a raising of the temperature of (or within) the integrated humidity and temperature sensor. In a still further aspect, raising the temperature includes use of a heating element included in the integrated humidity and temperature sensor. In one aspect, the integrated humidity and temperature sensor includes a processor device, whether microcontroller or hardwire implemented, that executes the methods of sensor calibration described above. In one aspect, the processor device is configured to raise the temperature by switching the heating element on, monitoring the temperature rise, and switching the heating element off upon reaching a pre-determined temperature rise. In another aspect, the second humidity value and the second temperature are measured immediately upon reaching the pre-determined temperature rise.

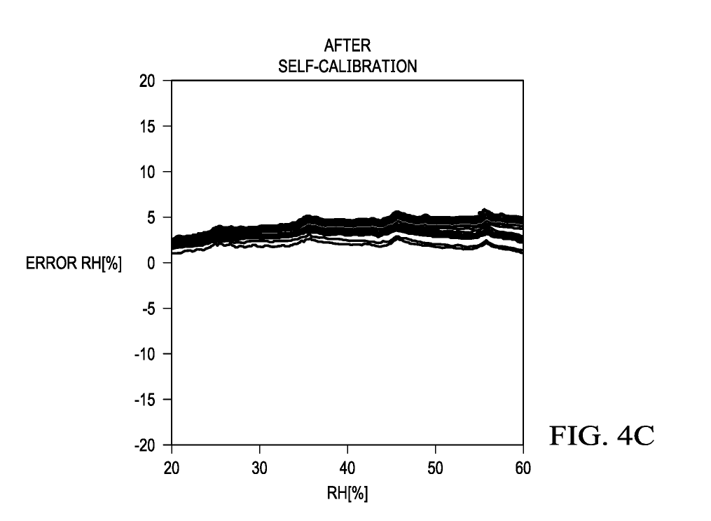

FIGS. 4A-4C illustrate before and after results of an example implementation of a method of calibrating a relative humidity sensor using an integrated humidity and temperature sensor. Thirty-two sensor units were involved in the example simulations illustrated. FIG. 4A illustrates percent error in relative humidity (Error RH[%]) of 32 fresh sensor units. FIG. 4B illustrates simulated aging of the 32 sensor units after exposing the units to 85%RH/85degC for 200 hours. In absence of self-calibration, FIG. 4B illustrates a resulting drift between 10% and 20%, where drift was determined using the above-identified, formula 2, drift relationship. The temperature increase, or step, used was 60 degrees Celsius. FIG. 4C illustrates the 32 sensor units after recalibration, where an output of the respective sensor unit is shifted by the amount of drift determined. FIG. 4C illustrates that, after implementation of the calibration method, the drift is almost completely recovered.

The resistive heating element included in the integrated humidity and temperature sensor used in the example implementation shown in FIGS. 4A-4C was capable, at highest supply, of delivering 360 mW to the respective sensor, increasing temperature of the sensor by tens of degC in few seconds. In the example implementation, 20 seconds of heating was found to be more than enough time to reach thermal equilibrium inside the respective sensor, and to let any moisture diffuse inside the relative humidity sensing element. Time should be, at least in first approximation, independent of the temperature step amplitude.

Certain factors can be considered when determining a temperature step amplitude to use (degC temperature rise), and a heating phase time (seconds of temperature rise). These factors include certain errors in the above-identified, formula (2), drift relationship, such as polynomial approximation of the relationship (Nth order); a difference between the effective RH sensor temperature and the measured RH sensor temperature due to the distance between the RH sensor and the temperature sensor; and any temperature sensor offset and/or gain error.

As shown in FIG. 1, the relative humidity sensing element 110 is located in the polyimide layer, outside the silicon circuit 140, with the temperature sensing element 130 in the active area, and the heating element 120 between the RH sensing element 110 and the temperature sensing element 130. Due to the thermal impedance of silicon, polyimide, air and the package at the steady state, the temperature measurement at the temperature sensor will be slightly below the actual temperature at the RH sensor. Part of this error can be digitally compensated, and part is a function of the polyimide layer thickness.

Regarding accuracy of a determination of drift, the temperature sensor error decreases as the temperature step increases, while the computational error due to polynomial approximation of the exponential is close to zero for very small temperature steps (where the exponential can easily be approximated by a first order equation). The impact of the temperature difference between the RH and temperature sensor becomes more and more relevant as the temperature step decreases; this error being the main error source for small temperature steps.

Accordingly, a main challenge to reducing the temperature step increase (in order to save power) is the systematic error due to the distance between the RH sensing element (i.e., the polyimide, on top of the passivation), the temperature sensing element (based on bipolar transistor, located in the active area) and the heating element.

While the disclosure has been described with reference to illustrative examples, this description is not intended to be construed in a limiting sense. Various other examples of the disclosure will be apparent to persons skilled in the art upon reference to this description.

Although method steps may be presented and described herein in a sequential fashion, one or more of the steps shown and described may be omitted, repeated, performed concurrently, and/or performed in a different order than the order shown in the figures and/or described herein. Accordingly, aspects described should not be considered limited to the specific ordering of steps shown in the figures and/or described herein.

It is therefore contemplated that the appended claims be interpreted to embrace all such variations and modifications of the aspects described.

What is claimed is:

1. A method of calibrating a humidity sensor, the method comprising the steps of:
measuring a first humidity value using a humidity sensor at a first time;
measuring a first temperature indicative of a temperature of the humidity sensor at the first time;
raising a temperature of the humidity sensor;
measuring a second humidity value using the humidity sensor at a second time;
measuring a second temperature indicative of a temperature of the humidity sensor at the second time;
determining a drift value of the humidity sensor according to the first and the second temperatures and the first and the second humidity values; and
adjusting the humidity sensor by the drift value.

2. The method of claim 1, wherein the raising of the temperature includes a temperature step profile selected from the group consisting of fixed time-variable temperature step and fixed temperature step-variable time.

3. The method of claim 1, wherein adjusting the humidity sensor includes shifting an output of the humidity sensor by the drift value.

4. The method of claim 1, wherein determining the drift value of the humidity sensor includes calculating a numerator over a denominator, where the numerator is a first value minus the second humidity value, where the first value is the first humidity value multiplied by a second value, the second value including a difference between the first temperature and the second temperature, and where the denominator includes the second value.

5. The method of claim 1, wherein determining the drift value of the humidity sensor includes calculating:
$$drift = \frac{{RHoM}^{eb} ( \frac{{T}_{0}}{c + {T}_{0}} - \frac{{T}_{1}}{c + {T}_{1}} ) - {RH}_{1 M}}{{e}^{b} ( \frac{{T}_{0}}{c + {T}_{ 0}} - \frac{{T}_{1}}{c + {T}_{1}} ) - 1}$$
where RH_{oM} is the first humidity value, RH_{1M} is the second humidity value, T₀ is the first temperature, T₁ is the second temperature, and b and c are constants.

6. The method of claim 5, wherein b is equal to 17.123 and c is equal to 234.95.

7. The method of claim 1, wherein the first and the second temperatures are indicative of the temperature of the humidity sensor within a vicinity of the humidity sensor.

8. The method of claim 7, wherein within the vicinity of the humidity sensor is within an integrated humidity and temperature sensor.

9. The method of claim 8, wherein raising the temperature includes use of a heating element included in the integrated humidity and temperature sensor, and measuring the first and the second temperature includes use of a temperature sensor in the integrated humidity and temperature sensor.

10. The method of claim 1, wherein pressure is constant at each of the first time and the second time.

11. An integrated humidity and temperature sensor, comprising:
a humidity sensor configured to output a signal corresponding to a first humidity value at a first time and a second humidity value at a second time;
a temperature sensor configured to output a signal corresponding to a first temperature at the first time and a second temperature at the second time;
a heating element configured to raise a temperature of the humidity sensor and the temperature sensor between the first time and the second time; and
a processor device configured to determine a drift value of the humidity sensor according to the first and the second temperatures and the first and the second humidity values.

12. The integrated humidity and temperature sensor of claim 11, wherein the processor device is further configured to adjust the humidity sensor by the drift value.

13. The integrated humidity and temperature sensor of claim 12, wherein, to adjust the humidity sensor by the humidity value, the processor device is further configured to shift an output of the humidity sensor by the drift value.

14. The integrated humidity and temperature sensor of claim 11, wherein, to determine the drift value of the humidity sensor, the processor device is further configured to calculate a numerator over a denominator, where the numerator is a first value minus the second humidity value, where the first value is the first humidity value multiplied by a second value, where the second value includes a difference between the first temperature and the second temperature, and where the denominator includes the second value.

15. An apparatus comprising:
a humidity sensor configured to output a signal corresponding to a humidity value;
a temperature sensor configured to output a signal corresponding to a temperature;
a heating element configured to raise a temperature of the humidity sensor and the temperature sensor; and
a processor device in communication with the humidity sensor, the temperature sensor, and the heating element, the processor device configured to:
measure a first humidity value at a first time;
measure a first temperature at the first time;
raise a temperature within the apparatus;
measure a second humidity value at a second time;
measure a second temperature at the second time; and
determine a drift value of the humidity sensor according to the first and the second temperatures and the first and the second humidity values.

16. The apparatus of claim 15, wherein the processor device is further configured to adjust the humidity sensor by the drift value.

17. The apparatus of claim 16, wherein, to adjust the humidity sensor by the drift value, the processor device is further configured to shift an output of the humidity sensor by the drift value.

18. The apparatus of claim 13, wherein, to determine the drift value of the humidity sensor, the processor device is further configured to calculate a numerator over a denominator, where the numerator is a first value minus the second humidity value, where the first value is the first humidity value multiplied by a second value, where the second value includes a difference between the first temperature and the second temperature, and where the denominator includes the second value.

19. The apparatus of claim 13, wherein, to raise the temperature within the apparatus, the processor device is further configured to switch the heating element on, monitor a temperature rise within the apparatus, and switch the heating element off upon reaching a pre-determined temperature rise.

20. The apparatus of claim 19, wherein the processor device is further configured to measure the second humidity value and the second temperature immediately upon reaching the pre-determined temperature rise.
